(19) United States
(12) Patent Application Publication
(10) Pub. No.: US 2004/0138138 A1
(43) Pub. Date: Jul. 15, 2004

(54) Use of LHRH-antagonists in doses that do not cause castration for the improvement of T-cell mediated immunity

(76) Inventors: Jurgen Engel, Alezenau (DE); Manfred Peukert, Oberursel (DE)

Correspondence Address: Goodwin Procter LLP, 599 Lexington Avenue, New York, NY, 10022, US

(21) Appl. No.: 10/748,887

(22) Filed: Jul. 30, 2002

Related U.S. Application Data
(60) Provisional application No. 60/309,735, filed on Aug. 2, 2001.

(51) Int. Cl. A61K 38/07

(52) U.S. Cl. 514/16; 514/17

(57) ABSTRACT

The invention concerns the use of appropriate doses of an LHRH-antagonist to lower sex hormone levels resulting in a modification of the T-cell population in an individual suffering from a disease that will respond favourably to such modification. A preferred LHRH-antagonist is cetrorelix.

Use of LHRH-antagonists in doses that do not cause castration for the improvement of T-cell mediated immunity

In a patent by R. L. Boyd (WO 200062657, AU 200037977) the autor claims that disrupting the sex steroid signalling by application of an LHRH-agonist will result in a modification of the T-cell population in subjects with a depressed or abnormal T-cell population. This treatment will have the undesired side-effect of castration of the subject, but the author claims that this castration will be reversible upon cessation of treatment.

This side effect is highly undesirable as it will result in loss or reduction of libido, sexual desire and sexual potency. In men and pre-menopausal women the treatment would also result in the typical symptoms of lowering the sex hormone-level below castration level, e.g. hot flushes, women will additionally be at risk to lose bone minerals, potentially limiting the duration of treatment.

These unwanted effects can be limited by using an LHRH-antagonist in a dose that will not result in castration but will still have the desired effect on the immune system.

The object has now been achieved in that an LHRH-antagonist is used for the production of a medicament for treating of an individual where the treatment results in a modification of the T-cell population in an individual suffering from a disease that will respond favourable to such a modification, suffering from a HIV-infection or cancer, or an auto-immune disease, or benign prostatic hyperplasia, or endometriosis, or asthma, or arthritis, or dermatitis, or multiple sclerosis, or Jacob Creuzfeldt-disease or Alzheimer disease, further to enhance the immun response to an antigen, to decrease the host versus graft reaction and to enhance the anti-aging-treatment.

The preferred LHRH-antagonist can be cetrorelix, teverelix, antide, abarelix.

Expediently, the medicament can be administered in the following ratio:

Total dose from 5 mg to 120 mg LHRH-antagonist, divided in a period of 1 to 8 weeks and according to needs with repeat of the therapy every 3 to 4 months.

It has been found a preferred embodiment of the therapy with the LHRH-antagonist cetrorelix.

Cetrorelix pamoate in a total dose from 30 mg to 120 mg divided in a period of 1 to 4 weeks and according to needs with repeat of the therapy every 3 to 4 months,

Cetrorelix acetate in a total dose from 5 mg to 80 mg divided in a period of 1 to 8 weeks and according to needs with repeat of the therapy every 3 to 4 months.

We checked the efficacy with a patient population of

140 elderly patients (older than 50 years) with benign prostatic hyperplasia

45 patients with endometriosis in which the immune cell suppression play a role.

1. Use of appropriate doses of an LHRH-antagonist, peptidic or non-peptidic, that will lower sex hormone levels to a certain extent but not below the castration level.

2. Use of appropriate doses of an LHRH-antagonist to lower sex hormone levels resulting in modification of the T-cell population.

3. Use of appropriate doses of an LHRH-antagonist to lower sex hormone levels resulting in a modification of the T-cell population in an individual suffering from a disease that will respond favourably to such a modification.

4. Use of appropriate doses of an LHRH-antagonist to lower sex hormone levels resulting in a modification of the T-cell population in an individual suffering from a HIV infection, cancer, an auto-immune disease, benign prostatic hyperplasia, endometriosis, asthma, arthritis, dermatitis, multiple sclerosis, Jacob Creuzfeldt-disease, Alzheimer's disease an for anti-aging-treatment.

5. Use of appropriate doses of an LHRH-antagonist to lower sex hormone levels resulting in a modification of the T-cell population resulting in an enhanced immune response to an antigen.

6. Use of appropriate doses of an LHRH-antagonist to lower sex hormone levels resulting in a modification of the T-cell population resulting in a decrease of host versus graft reaction.

7. Examples for substances that can be used as LHRH-antagonists according to claims 1-6 are cetrorelix, teverelix, antide, or abarelix.

8. Use of a LHRH-antagonist for producing a medicament for the treatment of diseases according to claims 1 to 7.

9. Use according to claim 8, characterized in that the LHRH-antagonist is administered in the following total dose from 5 mg to 120 mg divided in a period of 1 to 8 weeks and according to needs with repeat of the therapy every 3 to 4 months.

10. Use according to claims 8 and 9, characterized in that cetrorelix pamoate is administered in the following total dose from 30 mg to 120 mg divided in a period of 1 to 4 weeks and according to needs with repeat of the therapy every 3 to 4 months.

11. Use according to claims 8 and 9, characterized in that cetrorelix acetate is administered in teh following total dose from 5 mg to 80 mg divided in a period of 1 to 8 weeks and according to needs with repeat of the therapy every 3 to 4 months.
